(19) United States
(12) Patent Application Publication
(10) Pub. No.: US 2014/0134697 A1
(43) Pub. Date: May 15, 2014

(54) PROCESS FOR THE DIGESTION OF ORGANIC MATERIAL

(71) Applicants: Hendrik Louis Bijl, Echt (NL); Vincent Pascal Pelenc, Echt (NL)

(72) Inventors: Hendrik Louis Bijl, Echt (NL); Vincent Pascal Pelenc, Echt (NL)

(73) Assignee: DSM IP ASSETS B.V., Heerlen (NL)

(21) Appl. No.: 14/128,283

(22) PCT Filed: Jun. 26, 2012
(86) PCT No.: PCT/EP2012/062386

§ 371 Date: Dec. 20, 2013

(30) Foreign Application Priority Data
Jun. 29, 2011 (EP) 11171856.5
Jul. 28, 2011 (EP) 11175709.2

(51) Int. Cl. C12P 5/02

(52) U.S. Cl. CPC C12P 5/023
USPC 435/167

(57) ABSTRACT

The present invention provides a process for the digestion of organic material into biogas which comprises:
treating the organic material to reduce the number of viable microorganisms in the organic material;
treating the organic material with one or more enzymes;
separating the liquid fraction from the solid fraction of the enzyme-treated organic material; and
digesting the liquid fraction to form biogas.

### FIELD OF THE INVENTION

The present invention relates to a process to digest organic material.

### BACKGROUND OF THE INVENTION

The production of biogas via the anaerobic digestion of organic material is a rapidly growing source of renewable energy. The process is complex; a combined action of several biotechnological processes determines the stability, efficiency and yield of the biogas produced. An optimal process design is still under active research done at laboratory and pilot plants. Substrates like grass, manure or sludge can be used as feed for the biogas production due to their high yield potential.

The digesting systems are often divided in one-stage and two-stage digesters. In one-stage digesters all the microbiological phases of the anaerobic digestion takes place in one tank or fermenter. In the two-stage digesters the hydrolysis and acidification will take place in the first reactor and acetogenesis and methanogenesis occurs in the second reactor. The two phase concept is often chosen for optimisation of the digestion process in order to produce more methane. Both one-stage and two-stage processes have in common that all the phases of the anaerobic digestion are a microbiological process, involving the presence of suitable consortia of microorganisms.

There are several reactor configurations used for the production of biogas from all kind of waste (water) treatment systems: CSTR (continuously stirred tank reactor), SBR (sequential batch reactor), involves periodic settling phases that allow microorganisms to remain longer, and in this way uncouples the growth rate to the hydraulic retention time. Another option is to apply an anaerobic membrane bioreactor (AnMBR). More sophisticated systems are UASB (upflow anaerobic sludge blanket), EGSB (expended granular sludge bed) or IC (Internal Circulation) reactor are often used for the production of biogas in all kind of waste water treatment systems, designs that are directed to the optimisation of higher OLR (organic loading rate), reduced HRT (hydraulic retention time) and higher methane (biogas) yields.

### SUMMARY OF THE INVENTION

The present invention provides an improved process for the digestion of organic material in biogas. The process is a two-stage process whereby only the second stage is a microbiological digestion. In the first stage the organic material is heat treated to prevent growth of microorganisms present and the heat-treated organic material is enzymatically treated. The effluent of the first stage is separated in a liquid and in a washed solid fraction. The liquid fraction is fed to the second stage to produce biogas. The thermal and enzymatic treatment allows a control and an optimization of this step (first phase) which cannot be realized in an active microbiological environment. The process of the invention makes lower overall retention times possible without loss of gas yield compared to prior art processes.

### LEGEND TO THE FIGURE

FIG. 1 shows the variations in sequence and combinations of lytic enzymes to reduce the number of microorganisms in pig manure

### DETAILED DESCRIPTION OF THE INVENTION

By biogas is meant the product produced by the anaerobic digestion or fermentation of biodegradable materials. Biogas comprises primarily methane and carbon dioxide and may have small amounts of hydrogen sulphide, moisture and siloxanes. In special cases hydrogen is the targeted product.

By organic or biological material is meant matter that has come from a once-living or still-living organism; is capable of decay, or the product of decay. Preferably the organic material is microbial material such as sludge or biomass from purification, fermentation or digestion processes. Especially bacterial sludge from an aerobic purification process or bacterial biomass from an aerobic digestion can be advantageously treated according to the present invention.

By sludge or activated sludge is meant the solid waste or solid waste product or solid biomass of waste water or sewage treatment. This solid waste product consists mainly of bacteria. Preferably sludge of an aerobic purification step or system is used. Suitable organic waste streams that can be used in the present process are fermentation broths or fractions thereof from industrial fermentation industries. Another suitable organic waste stream is manure such as cow, pig, goat or horse manure.

By organic matter content of the organic material is meant the dry matter content of the organic material minus ash. COD (Chemical Oxygen Demand) test is commonly used to indirectly measure the amount of organic matter content of the organic material, see for example ISO 6060 (1989).

The present invention provides a process for the digestion of organic material into biogas which comprises:
treating the organic material to reduce the number of viable microorganisms in the organic material;
treating the organic material with one or more enzymes;
separating the liquid fraction from the solid fraction of the enzyme-treated organic material; and
digesting the liquid fraction to form biogas.

The process of the invention is capable to treat all kind of digestible organic material. The separation of the enzymatic step and the microbiological digestion allows an optimal control and the selection of conditions to treat the organic material. Examples of suitable substrates are energy crops like grass, farm waste like manure or agricultural waste, sludge from waste water treatment systems, the organic fraction of municipal waste, biomass from fermentation industries and bio refineries. Also mixes of several organic materials can be used in the process of the invention

The process of the invention is capable to treat all kind of digestible organic material such as sludge or other organic material, preferably bacterial sludge or other bacterial organic waste. Especially bacterial sludge from an aerobic purification process or bacterial biomass from an aerobic digestion can be treated according to the present invention. The bacteria of these aerobic processes are found to be enzymatically digestible. The cell walls of these bacteria are found to be degradable by lytic enzymes optionally in combination with the pretreatment of the sludge or biomass as described herein. The separation of the enzymatic step and the optional microbiological digestion provides for an optimal control and selection of conditions to treat the organic material. Also fractions of sludge or mixes of several kinds of sludges or fractions thereof can be used in the process of the invention. Moreover sludge may be mixed or combined with other organic material or substrates like grass or manure. Apart from sludge also other microbial material such as biomass originating from for example yeast or fungal fermentation industries such as breweries or algae biomass from the cultivation of algae, can be used in the process of the present invention. The present process is found to be very useful for N-enriched substrates or digestible organic material. The organic material is preferably heat-treated or pasteurized at a temperature of 65 to 120° C., more preferably at 65 to 95° C. for a suitable time. Pasteurization is a process of heating the organic material to a specific temperature for a definite length of time in a humid environment. For example pasteurization at 72° C. for 30 seconds is sufficient. For example 1 hour at 120° C. gives the same results as 4 hours at 90° C. with respect to the CFU count (see below). In general high temperatures may result in more protein denaturation as well as occurrence of toxic compounds. In general if the pasteurization time is longer, the pasteurization temperature can be lower. Water content at pasteurization should be sufficient to enable pasteurization effect. In general the water content is between 30 and 95 wt %, preferably between 50 and 90 wt %. This process slows microbial growth in the organic material. Pasteurization or heat-treatment is not intended to kill all micro-organisms in the organic material. Instead pasteurization or heat-treatment aims to reduce the number of viable microorganisms so they are unlikely to substantially produce biogas or other fermentation products like organic acids and alcohols, in the first stage (or first step or first phase or enzyme treatment) of the process. In general in the first stage less than 2%, preferably less than 1%, of the total of biogas is formed. After the pasteurization or heat-treatment according to the invention the CFU count is in general lower than 10⁶, preferably less than 10⁵, even more preferably less than 10⁴ and most preferably less than 10³ CFU/ml in the organic material present. In microbiology, colony-forming unit (CFU or cfu) is a measure of viable bacterial or fungal numbers. Unlike direct microscopic counts where all cells, dead and living, are counted, CFU measures viable cells. The pasteurization step also facilitates the use of enzymes or enzyme mixtures directly originating from harvested enzyme production fermentations.

Another way to characterize the efficacy of a treatment that reduces in the number of microorganisms is by calculating the logarithm of the number of CFUs of the starting material divided by the number of CFUs of the material after the treatment. The advantage of this method is that—since the killing of micoorganisms is generally assumed to be a first-order reaction—the log reduction of a treatment is largely independent of the actual number of microorganisms present. A sterilization procedure may be required to deliver as much as log 10 reduction (which would kill off as many as 10⁸ microorganisms or more), but in the case of the present invention such high efficacy is not required, or not even desirable. An effective treatment procedure in the present invention would deliver at least a log 1 reduction in the number of CFUs, preferably log 2, even more preferably log 3.

In general, it is beneficial for the process to have the thermal treatment at low or high pH, for example a low pH treatment at pH<4, more preferably at pH<3, even more preferably at pH<2, the low pH treatment is in general done at pH>−1, or for example a high pH treatment at pH>8, more preferably pH>9, even more preferably pH>10. The advantages of thermal treatment at high and low pH's, are for example solubilization and partial hydrolysis of polymers, such as proteins, carbohydrates, such as starch as hemicellulase, and lipids, but also the reduction of viable cells will be enhanced by extreme pH's, resulting in for example a need of lower temperature and/or less time for the thermal treatment. Additional advantages of high pH treatment are for example improving solid/liquid separation at the end of the thermal and enzyme treatments, improved solubilization of protein and fat, and ammonia stripping for feedstocks having high ammonia content. Chemicals to be used for adjustment of the pH can be for example hydrochloric acid, phosphoric acid, and sulphuric acid for lowering the pH, or for increasing the pH potassium hydroxide and sodium hydroxide.

According to the present invention hardly any biogas is formed during the enzyme treatment of the organic material and the biogas production takes place in the biogas fermenter. Another advantage of the present process is that the enzymes used are hardly inactivated or consumed by microorganisms present. The low numbers of viable microorganisms present have hardly any effect on the enzymes added and their activity.

The heat-treatment needs the addition of energy to the organic material. It is noticed that the addition of this energy is compensated by an increased biogas production compared to the situation without this heat-treatment. In most cases even more energy is produced in the form of biogas than is needed for the heat-treatment.

Optionally before, during or after the pasteurization or heat-treatment (part of) the organic material can be pre-treated to make for example the material such as the cellulose present more accessible to the enzymes. The pretreatment can for example be a mechanical, chemical or thermal pretreatment or a combination thereof. A steam explosion treatment or a high temperature treatment of more than 120° C. are examples of thermal treatment. Chemical oxidation or chemical hydrolysis (for example using strong an acid or alkaline compound) can be used as chemical pretreatment. Ultrasonic treatment or grinding (or blending or homogenizing) are examples of mechanical pretreatments. To the temperature treated organic material one or more enzymes are added. Said enzyme(s) make(s) the degradation of organic material possible in the pasteurized or heat-treated medium, in which microbial growth is limited. This will result in an improved biogas production compared to a process wherein no enzyme is used. In general more than one enzyme is used, advantageously an enzyme composition comprising at least a protease and/or a cellulase, preferably at least a protease, a lipase and a cellulase, and optionally an amylase, a hemicellulase, a phytase and/or a lysing enzyme is used. The enzymes decompose the long chains of the complex carbohydrates, proteins and lipids into shorter parts. For example, polysaccharides are converted into oligosaccharides and/or monosaccharides. Proteins are split into peptides and amino acids. Also other enzyme compositions can be used which promote the degradation of the organic material. The enzymes can be mixed to form the selected combination or can be produced as a mixture by a selected strain during selected fermentation conditions. For example the enzyme mixture obtained from a fermentation broth of a fungus such as Trichoderma, Aspergillus or Talaromyces or a bacterium such as Bacillus can be used. The enzyme mixture can be designed in relation to the composition of the substrate or organic material added. For example in case high amounts of fatty material are present, lipase can be added to the process, in case carbohydrates are present, amylase can be included in the enzymes used.

The enzyme step can be done as a one-step or a multi-step process. A multi-step process allows the optimization of the process for, for example, the properties of the enzymes. So the cellulase and protease treatment can be done separately, or a cellulase and protease treatment can be repeated.

Preferably one of the enzymes used is thermostable. Preferably, the activities in the enzyme composition may be thermostable. Herein, this means that the activity has a temperature optimum of 60° C. or higher, for example 70° C. or higher, such as 75° C. or higher, for example 80° C. or higher such as 85° C. or higher. All activities in the enzyme composition will typically not have the same temperature optima, but preferably will, nevertheless, be thermostable.

Cellulases are enzymes that hydrolyze cellulose (β-1,4-glucan or β D-glucosidic linkages) resulting in the formation of glucose, cellobiose, cellooligosaccharides, and the like. Also cellulase-enhancing proteins such as GH61 are comprised by the term cellulase herein.

Cellulases have been traditionally divided into major classes: endoglucanases (“EG”, (E.C. 3.2.1.4), which hydrolyze the beta-1,4-linkages between glucose units) (EC 3.2.1.4) (“EG”), exoglucanases or cellobiohydrolases (“CBH”, (E.C. 3.2.1.91), which hydrolyze cellobiose, a glucose disaccharide, from the reducing and non-reducing ends of cellulose) and β-glucosidases ([β]-D-glucoside glucohydrolase (“BG”, (E.C. 3.2.1.21), which hydrolyze the beta-1,4 glycoside bond of cellobiose to glucose). See e.g. Knowles et al., TIBTECH 5, 255-261, 1987; Shulein, Methods Enzymol., 160, 25, pp. 234-243, 1988. Endoglucanases act mainly on the amorphous parts of the cellulose fibre, whereas cellobiohydrolases are also able to degrade crystalline cellulose (Nevalainen and Penttila, Mycota, 303-319, 1995). Thus, the presence of a cellobiohydrolase in a cellulase system is required for efficient solubilization of crystalline cellulose (Suurnakki, et al. Cellulose 7:189-209, 2000). β-glucosidase acts to liberate D-glucose units from cellobiose, cello-oligosaccharides, and other glucosides (Freer, J. Biol. Chem. vol. 268, no. 13, pp. 9337-9342, 1993).

Proteases (protein degrading or modifying enzymes) are for instance endo-acting proteases (serine proteases, metalloproteases, aspartyl proteases, thiol proteases), exo-acting peptidases that cleave off one amino acid, or dipeptide, tripeptide etceteras from the N-terminal (aminopeptidases) or C-terminal (carboxypeptidases) ends of the polypeptide chain.

Lipases or fatty material splitting enzymes are for instance triacylglycerol lipases, phospholipases (such as A₁, A₂, B, C and D) and galactolipases.

Hemicellulase is a collective term for a group of enzymes that break down hemicellulose. Examples are xylanase, β-xylosidase, a-L-arabinofuranosidase, a-galactosidase, acetyl esterase, β-mannosidase and β-glucosidase.

A phytase (myo-inositol hexakisphosphate phosphohydrolase) is any type of phosphatase enzyme that catalyzes the hydrolysis of phytic acid (myo-inositol hexakisphosphate) which is an indigestible, organic form of phosphorus that is found in grains and oil seeds, and releases a usable form of inorganic phosphorus.

By lysing or lytic enzyme is meant an enzyme that is capable of lysis of the cell wall of a microorganism. Microorganisms include bacteria, fungi, archaea, and protists; algae; and animals such as plankton and the planarian. Preferably the microorganism is a bacterium, fungus, yeast or alga. A microorganism or microbe is an organism that is unicellular or lives in a colony of cellular organisms.

The lysing enzyme can for example be a protease. Examples of lysing enzymes are lysozyme or muramidase (from chicken egg), lysing enzyme from Trichoderma harzianum, lytic enzyme from Lysobacter such as Lysobacter enzymogenes, lyticase from Arthrobacter luteus and Mutanolysin from Streptomyces globisporus ATCC 21553, labiase from Streptomyces fulvissimus or lysostaphin from Staphylococcus. Examples of lysing enzymes can for example be purchased from Sigma-Aldrich®. By the lysis of sludge or other bacterial materials the cell contents of the cells are liberated which make enzymes present in the sludge cells or bacterial material available for use in the process of the invention on top of the added enzymes. The liberated enzymes may even contain lytic enzymes which may lyse other bacteria.

The organic material is preferably pasteurized or heat-treated at a temperature of 65 to 120° C., more preferably 65 to 95° C. and therefore the enzyme has preferably an optimal activity at the pasteurization or heat-treatment temperature or at 0 to 10° C. lower, so in general at a temperature of 55 to 90° C. The enzyme is preferably stable at the selected pasteurization or heat-treatment temperature for a sufficient time, so for example for at least 30 minutes, preferably at least for 1 hour. By stability is meant that after a certain period of incubation time activity under the reaction condition is at least half of the starting activity.

The pH during the enzyme treatment will be in general between 4 and 9, preferably between 4 and 8, more preferably between 5 and 8. The enzyme is preferably stable at the selected pH for a sufficient time, so for example for at least 30 minutes, preferably at least for 1 hour.

The use of enzymes for hydrolysis of organic material containing polymer substrates was found to produce a high extraction yield of the organic matter in the liquid phase, in general higher than 75%. For (pig) manure or sludge this will be higher than 40%. The enzymes hydrolyze water-binding structures (proteins, polysaccharides), without producing polymer structures by themselves, like microorganism do. The enzymes produce the reduction of viscosity of the phases, what facilitates solid/liquid separation. Another way in which the enzymes can facilitate solid/liquid separation is by lowering the emulsification properties. For example proteases and lipases are known to be helpful in this respect. In this way the volume of solid phase is reduced. Processing and disposal of solid residues, which are important cost factors in waste water plants, are facilitated and cheaper. It was found that these properties of the enzymatic process simplify largely the solid/liquid separation and are surprisingly very advantageous in comparison to processes based on microbial hydrolysis. Moreover, the ability in the present invention of extracting with high yield the organic matter in soluble form is advantageous for the processing of this organic matter to biogas. Using such soluble substrate, it is possible to apply the technology of high load anaerobic reactors based on biomass retention (for example UASB and EGSB). This technology is not applicable in conventional processes using partially and slowly degradable insoluble substrates.

The pasteurization or heat-treatment step can be done before or (partly) during the enzyme treatment. In case the pasteurization or heat-treatment (partly) coincides with the enzyme treatment, the pasteurization or heat-treatment time may be as long as the enzyme treatment time. The enzyme treatment time will depend on for example the temperature used, the substrate, the enzymes(s) used, and the concentration of the enzymes. In general the enzyme treatment will take 2 to 50 hours, preferably 3 to 30 hours. The enzyme treatment can be batch wise or continuously, for example a CSTR reactor can be used.

Optionally at the end of the enzyme treatment the treated organic material is treated to deactivate at least part of the enzyme(s) present. For example a heat shock, a pH change can be applied. Also the enzymes used may be selected to become de-activated during the enzyme treatment process after the enzymes have fulfilled their job. In general the enzymes used, are chosen not to have a substantial negative effect on the biogas production later on in the process or even to contribute in a positive way in the biogas production phase.

In the solid/liquid separation step the liquid fraction is separated from the solid fraction of the treated organic material. Preferably optimal conditions are chosen during the solid/liquid separation such as pH, temperature, addition of flocculants or filter aids etc. All kinds of suitable separation techniques can be used such as decantation, filtration, centrifugation or combinations thereof. Optionally flocculant or filter aid is added before the separation takes place in order to improve the separation. Especially flocculants and filter aids which are biologically degradable such as cellulose are advantageously applied. To prevent loss of soluble digestible material the obtained filter cake or centrifuge sludge may be washed. The wash liquor is combined with the primary obtained filtrate or supernatant. To perform these process steps at the enzyme incubation temperatures will facilitate the separation process.

The solid fraction from the solid/liquid separation can be processed or used for example by incineration (combustion), composting or spreading on cultivated areas, or forests. The present process having a temperature treatment step, allows composting or spreading of the solid fraction without a further thermal treatment of the solid fraction which is often required in case of spreading of sludge or other biomass.

During the enzyme treatment and/or separation step anaerobic or aerobic conditions can be maintained. In general no special measures have to be taken to keep anaerobic conditions.

The liquid fraction from the solid/liquid separator is introduced in biogas reactor. Upflow anaerobic filters, UASB, anaerobic packed bed and EGSB reactors are examples of high-rate digesters on industrial scale. Especially UASB and EGSB reactors offer benefits of high-rate digesters when applied at high organic loading rates. The use of liquid and solubilized substrate in the biogas reactor enables a very high loading of the reactor. In general 2 to 70 kg COD/m³/day, preferably at least 10 COD/m³/day and/or less than 50 kg COD/m³/day can be introduced in the biogas reactor. More preferably at least 20 kg COD/m³/day can be introduced in the biogas reactor. Preferably the HRT in the EGSB digester is between 3 to 100 hours, more preferably between 3 and 75 hours, even more preferably between 3 and 60 hours and most preferably between 4 and 25 hours. Preferably the HRT in a IC reactor is between 3 to 100 hours, more preferably between 10 and 80 hours and most preferably between 15 and 60 hours. Preferably the HRT in the UASB digester is between 10 to 100 hours, more preferably between 20 and 80 hours and most preferably between 20 and 50 hours. Preferably the HRT in the CSTR digester is between 1 to 20 days, more preferably between 2 to 15 days and most preferably between 2 to 10 days. In general no recycling of liquid to the first stage (enzyme treatment) will take place. In a CSTR system measures can be taken to keep the biomass in the reactor. Preferably the HRT in the anaerobic membrane bioreactor is between 3 to 12 days, more preferably between 4 and 10 days.

In recycling liquid, microorganisms will be present that will start producing biogas in the first phase in case of liquid recycling. If recycling of liquid is desired, measures have to be taken that no biogas production will occur in the first phase due to introducing anaerobic microorganisms, for example the recycling liquid can be pasteurized or sterilized.

The pH of the biogas reactor will in general be between pH of 3 and 8, preferably between pH of 6 and 8. Generally no measures have to be taken to control the pH, the system is capable to maintain this pH itself. In case the substrate of the biogas reactor is outside this pH range, so for example at pH of 5 or lower, or at pH of 9 and higher, the pH of this substrate is preferably neutralized to for example between 6 and 8.

The process of the invention is directed to an optimal use of the energy that is applied for the thermal treatment of the organic material. Directly applying the next steps of the process of the invention may reduce energy losses in the form of heat that is lost in the enzymatic treatment, liquid/solid separation and biogas production. The enzymatic treatment, liquid/solid separation and biogas production may take place at temperatures almost the same as the thermal treatment temperature without the addition of extra heating or other forms of energy supply. Therefore the solid/liquid separation preferably takes place at 70 to 50° C. The biogas production preferably takes place at 65 to 30° C. and most preferably takes place at 65 to 40° C.

The process of the invention can be performed in many ways including batch, fed batch or continuously loaded reactors or fermenters or a combination thereof. For the enzymatic treatment batch reactors are preferred. In the biogas production phase continuous reactors like UASB or EGSB are preferred.

### EXAMPLES

### Methods and Materials

### Enzymes

Enzymes used for the incubations of the various feedstocks were commercially available enzyme samples of the classes of hemicellulases, cellulases, proteases and lysozyme. The hemicellulase product used was Bakezyme® ARA10.000, the cellulase product was Filtrase® NL, the lysozyme product was Delvozyme® L, and the protease product applied was Delvolase®, a bacterial protease. All enzyme products are produced by DSM Food Specialties.

### Method for Determination of CFU

CFU is determined for Aerobic count using NEN-EN-ISO 4833:2003. For Anaerobic count NEN 6813:1999 is used.

### Method for Determination of Total Protein Content of Enzyme Solutions

The method was a combination of precipitation of protein using trichloro acetic acid (TCA) to remove disturbing substances and allow determination of the protein concentration with the colorimetric Biuret reaction. In the Biuret reaction, a copper (II) ion is reduced to copper (I), which forms a complex with the nitrogens and carbons of the peptide bonds in an alkaline solution. A violet color indicates the presence of proteins. The intensity of the color, and hence the absorption at 546 nm, is directly proportional to the protein concentration, according to the Beer-Lambert law. The standardisation was performed using BSA (Bovine Serum Albumine) and the protein content was expressed in g protein as BSA equivalent/L or mg protein as BSA equivalent /ml. The protein content was calculated using standard calculation protocols known in the art, by plotting the OD₅₄₆ versus the concentration of samples with known concentration, followed by the calculation of the concentration of the unknown samples using the equation generated from the calibration line.

Determination of Chemical Oxygen Demand (COD), Total COD, Soluble COD, Total Solids (TS), Total Suspended Solids (TSS), Ash, Volatile Solids (VS) (=Organic Dry Matter), Volatile Suspended Solids (VSS, are an Indication of the Maximum Cell Biomass Present in the System), Solubilization Yield, Total Kjeldahl Nitrogen, Ammonia Nitrogen

These parameters were determined according to standard procedures known in the art and as described by Standard Methods of American Public Health Association (APHA, 1995).

Biogas composition (CH4 and CO2) was measured using gas chromotography, equipped with a thermal conductivity detector (TCD). Volatile fatty acids (VFA) and ethanol concentration were measured using a gas chromatograph (Shimadzu GC-2010 AF, Kyoto, Japan), equipped with a flame ionization detector (FID) (Angelidaki et al., 2009).

The solubilization yield was determined using the organic dry matter content of the supernatant and the total slurry after pretreatment, using the following equation:

$$solubilization yield \% = \frac{\% {oDM}_{S}}{\% {oDM}_{T}} \times F \times 100 \%$$

$$in which \text{:} {oDM}_{S} = organic dry matter content of the supernatant$$

$${oDM}_{T} = organic dry matter content of the total slurry$$

$$\begin{matrix}F = correction \text{-} factor for the pellet \text{-} volume \\ = \frac{( 100 - \% {oDM}_{T} )}{( 100 - \% {oDM}_{S} )}\end{matrix}$$

The total protein content was calculated from the total Kjeldahl nitrogen content multiplied by 6.25. Except for pig manure, for which the content of ammonia nitrogen was subtracted from the total Kjeldahl nitrogen, followed by multiplication with 6.25.

### Method for Determination of Lipids

The sample is weighed into a suitable vial and lyophilized. After lyophilization, the weight is recorded. The dried residue is homogenized and approximately 1 gram is weighed into an extraction shell (type Whatman cellulose extraction thimbles 26 mm×60 mm single thickness). This shell is boiled in dichloromethane (Merck for liquid chromatography quality) for 1.5 hour in a Soxtec exctraction unit (Soxtec system MT 1043 extraction unit), at 119° C., using a pre-weighed appropriate Soxtec Cup. Then, the shell is refluxed for 1 hour. Subsequently the dichloromethane is evaporated.

The increase in weight of this cup is the fat content extracted from approximately 1 gram dried material. After correction for the dry weight determined during the lyophilisation step, the fat content per sample as such is expressed in g/Kg.

### Method for Determination of Carbohydrates and Lignin

Carbohydrates and lignin content were determined according to “Determination of structural carbohydrates and lignin in biomass”, A. Sluiter et al. Technical report NREL/TP-510-42618.

### Example 1

### Effect of Pretreatment Conditions and Enzyme Incubation on Solubilization of Brewers Spent Grain

Brewers spent grain (BSG), as milled and dried material, was obtained from a commercial brewery. The material was suspended in distilled water to a dry matter content of 10%, in a double-walled closed glass reaction chamber, which is connected to a circulating water bath, in which the water temperature was set to the desired temperature, i.e. 70° C. or 90° C. The pH of the suspension as such was pH 6.6, and was adjusted to pH 1.5, 4, 11.5, using 4N HCl or 4N NaOH. Subsequently, the slurries were incubated for certain time periods, while stirred. Alternatively, the BSG suspension was adjusted to the desired pH as described before, and treated in a flask in a sterilizer for the desired period of time, assuming a temperature of 120° C. After the different pretreatments, the slurries were cooled down, adjusted to pH 7.5 using HCl or NaOH, and incubated for 4 h at 60° C., with the addition of 100 mg Delvolase® per g BSG dry matter. Subsequently, the incubate was cooled down, pH adjusted to pH 5.0, and further incubated at 50° C. for 24 h, with the addition of 7.5 mg protein derived from Bakezyme® ARA10.000 per g BSG dry matter and 9 mg protein derived from Filtrase® NL per g BSG dry matter.

Prior to and after the 2 subsequent enzyme incubations a sample for each treatment was analysed for organic dry matter, which was then recalculated to solublization yield as described before. Results of solubilization yield after the whole series of treatment and enzyme incubations are presented in Table 1.

TABLE 1
Solubilization yields of BSG after pH, temperature treatment
combined with enzyme incubations, at different settings of pH,
temperature and time, as indicated, and as expressed as %.

| | 4 h 70° C. | 1 h 90° C. | 4 h 90° C. | 20 min 120° C. |
|---|---|---|---|---|
| pH 1.5 | 65 | 67 | 65 | 66 |
| pH 4 | n.d. | n.d. | 40 | 47 |
| pH 6.6 | 45 | 48 | 50 | 47 |
| pH 11.5 | 64 | 67 | 64 | 62 |

n.d. = not determined

From these results it is clear, that at neutral conditions 40 to 50% of the organic dry matter can be solubilized, whereas 60 to 70% can be solubilized at the more extreme pH conditions of pH 1.5 and 11.5. The contribution of the enzyme incubations was derived from the solubilization yield prior to the enzyme incubations, which was only in the range of 10-20% for the 4 h 70° C. pretreatments, and which amounted to 15-40% for the 4 h 90° C. and 20 min 120° C. pretreatments. The lower numbers of these ranges mentioned were found at neutral pH, whereas the upper limits were determined for the treatments at the more extreme pH conditions.

Further tests of these materials for gas yields confirmed the solubilization yields as shown in the table, demonstrating the highest gas yields for the samples with the highest solubilization yields.

### Example 2

### Pretreatment of Brewers Spent Grain at Pilot Scale

Brewers spent grain (BSG), as wet residu from the beer brewery process, was obtained from a commercial brewery. The composition of this material is presented in Table 2.

TABLE 2
Composition of wet BSG, as expressed in g/kg.

| | Content (g/kg) |
|---|---|
| Dry matter | 199 |
| Organic dry matter | 191 |
| Ash | 9 |
| Protein | 57 |
| Lipids | 15 |
| Lignin | 29 |
| Carbohydrates | 84 |

For producing sufficient quantity of solubilized BSG for anaerobic fermentation experiments, 400 kg of wet BSG was loaded into a stainless steel tank reactor with a gross volume of 1500 L, with the addition of 400 L water. The reactor was equipped with a cooling/heating jacket, in which 0.5 bar steam was used to heat the slurry to 90-95° C. While heating, the slurry of BSG was stirred with a variable speed anchor type mixer, at a stirrer speed of 50-55 rpm. The pH was adjusted to pH 10.7 by addition of 18 kg 25% NaOH solution. The slurry was then incubated at 90° C. at a stirrer speed of 40 rpm. After 4 h of incubation the pH of the slurry had decreased to pH 8.5, and the slurry was cooled to 62-65° C., using cold water in the jacket of the reactor. Subsequently, 8 kg of Delvolase® was added to the mixture, which was then allowed to incubate for 4 h at 60-62° C. at a stirrer speed of 25-30 rpm. After this incubation the pH had decreased to 7.4, and by the addition of 8.8 kg 30% HCl the pH was adjusted to pH 4.5. The slurry was then cooled to 50-52° C., and 600 g of protein derived from Bakezyme® ARA10.000 and 720 g protein derived from Filtrase® NL, each of them in a total volume of 8 kg solution, were added for further incubation of the mixture for 20 h at 42-45° C., at the indicated stirrer speed. Finally, the pH of the slurry was adjusted to pH 7.4 by the addition of 10.2 kg 25% NaOH.

Before further processing this slurry, 65 kg of the material was withdrawn for testing the fermentation capacity of the suspension. 731 kg of residual slurry was filtered in 2 parts, using a plate and frame membrane filter press with a cake volume of 180 L, and equipped with a multifilament cloth (Sefar Tetex multifilament 05 6456K). The first part of the slurry 386 kg was filtered at 1-2 bar. The cake was squeezed by inflating the membranes. Subsequently, the cake was washed in the filter with 200 kg of water. Primary filtrate, 165 kg, and washing liquor, 330 kg, were collected in separate tanks. The second part of the slurry was filtered similarly as described for the first one, except for the washing, which was omitted in the second cycle. This second cycle resulted in a primary filtrate of 345 kg. The combined primary filtrates, amounted to 510 kg, which was mixed with 100 kg of the washing liquor, resulting in a total amount of 610 kg of final filtrate for fermentation experiments.

The aerobic total plate counts of the starting slurry, and the slurry after the final enzyme incubation with Bakezyme® ARA10.000 and Filtrase® NL were determined and showed to have decreased from >1·10⁸ CFU/ml in the starting slurry, to 100 CFU/ml after the final enzyme treatment.

The composition of the slurry before filtration, and of the primary filtrate and the final filtrate (combination of primary filtrate with a portion of the washing liquor) is given in Table 3.

TABLE 3
Composition of the slurry before filtration, and of the primary
filtrate and final filtrate, which is a combination of primary filtrate
with a portion of the washing liquor, as expressed in g/kg.

| | Slurry before filtration | Primary filtrate | Final filtrate |
|---|---|---|---|
| Dry matter | 100 | 75 | 66 |
| Ash | 14 | 12 | 10 |
| Organic dry | 76 | 63 | 56 |
| matter | | | |
| COD | 149 | 86 | 77 |
| Protein | 26 | 23 | 20 |

### Jep

### Example 3

### Effect of Different Combinations of Lytic Enzymes in Pretreatment on Sanitation of Pig Manure

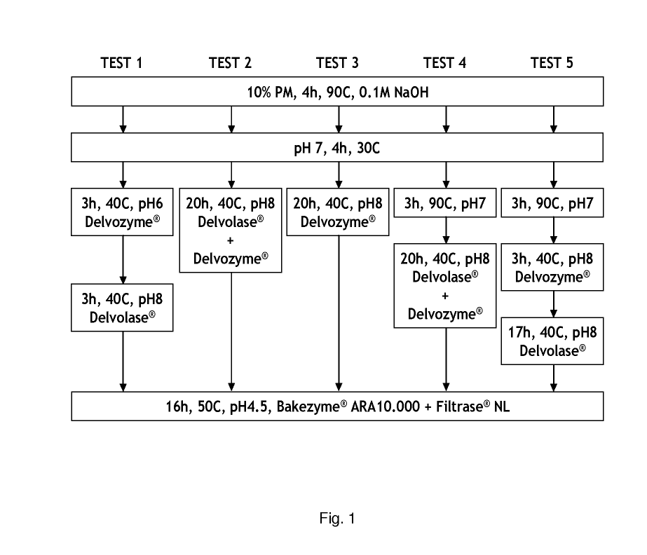

Fresh pig manure, with a dry matter content of approximately 10%, and pig manure concentrate, with a dry matter content of approximately 30%, were mixed at a ratio of 1:1, with 0.1M NaOH to a final dry matter concentration of 10%. Both fractions of pig manure were obtained from a manure trader. The pig manure concentrate was obtained according to a process as described in “Conversion to manure concentrates, Kumac Mineralen—Description of a case for handling livestock manure with innovative technology in the Netherlands”, Baltic Compass Report, 2011). Approximately 1 L of this mixture was heated to 90° C., while mixed, in a set-up as described in Example 1. Portions of approximately 100 ml of the cooled pretreated slurry were transferred to similar small-scale double walled reaction chambers, and different enzymes and treatments were tested with regard to the reduction of the number of microorganisms. The different tests performed are described in FIG. 1. The dosages of enzymes applied were similar as described in Example 1, and the dose of Delvozyme® L was 50 mg enzyme product per g pig manure dry matter.

The plate counts of the slurries prior to the Bakezyme® ARA 10.000 and Filtrase® NL incubation, are listed in Table 4.

TABLE 4
Aerobic total plate counts of the pig manure slurries after
finalization of the lytic enzyme incubation, i.e. prior to the
Bakezyme ® ARA 10.000 and Filtrase ® NL incubation,
at the different settings of test 1 to test 5, as shown in FIG. 1,
and as expressed as colony forming units per g (CFU/ml).

| | CFU/ml |
|---|---|
| Test 1 | >1.0 · 10⁶ |
| Test 2 | 1.1 · 10⁵ |
| Test 3 | 1.1 · 10⁵ |
| Test 4 | 1.6 · 10² |
| Test 5 | 3.8 · 10³ |

From this Table it is clear that the best reduction in aerobic total plate count is achieved by a combination of heat treatment, germination of surviving spores in a pH7 30° C. treatment, and finally a combined protease and lysozyme incubation. The introduction of the step to germinate surviving spores of 90° C., 4 h treatment shows to be very effective as is clear from the results of tests 1 to 3, versus tests 4 and 5. As known for pig manure the aerobic plate count as such is as high as >1.0·10¹¹ all treatments give a strong reduction, but Test 4 and 5 are preferred for efficient biogas production.

### Example 4

### Effect of Pretreatment Conditions and Enzyme Incubation on Sanitation and Solubilization of Pig Manure

Pig manure concentrate of approximately 30% dry matter was diluted in 0.1M NaOH to a dry matter content of 10%. A similar set-up as described in Example 1 was used to compare 2 different pretreatment methods. Both methods started with a 4 h incubation at 90° C. Subsequently, the slurry was cooled, pH adjusted to pH 7, and incubated for 4 h at 30° C., this was shown in Example 4 to be very beneficial for reduction of the number of microorganisms in the slurry. In method A, the slurry was then incubated for 3 h at 90° C., cooled down, adjusted to pH 8, and incubated for 20 h with the addition of Delvolase® and Delvozyme® L at 40° C., next pH was adjusted to pH 4.5, Bakezyme® ARA10.000 and Filtrase® NL were added, and the slurry was incubated for another 20 h at 40° C. In method B, the 3 h 90° C. incubation at pH 7 was performed after the Delvolase® and Delvozyme® L incubation. The remaining 20 h incubation with Bakezyme® ARA10.000 and Filtrase® NL was then performed after cooling down from 90° C. and pH adjustment to pH 4.5. The amounts of enzymes added per g dry matter of the pig manure was similar as stated in Example 1, for Delvolase®, Bakezyme® ARA10.000 and Filtrase® NL. The dose of Delvozyme® L was 50 mg enzyme product per g pig manure dry matter. For pH adjustments 4N NaOH or 4N HCl were used.

The reduction in the number of microorganisms in the pig manure in the different steps of the pretreatment is presented in Table 5.

TABLE 5
Total aerobic plate counts of the pig manure after finalization
of each of the different steps in the pretreatment process.

| Process step | Method A | Method B |
|---|---|---|
| 4 h 90° C. 0.1M NaOH | 3.3 · 10⁴ | 2.0 · 10³ |
| 4 h 30° C. pH 7 | 6.1 · 10⁴ | 6.0 · 10³ |
| 3 h 90° C. pH 7 | 8.0 · 10³ | n.d. |
| 20 h Delvolase ® + Delvozyme ® L pH 8 | 1.1 · 10⁵ | 45 |
| 3 h 90° C. pH 7 | n.d. | 40 |
| 20 h Bakezyme ® ARA10.000 + Filtrase ® NL | 3.0 · 10³ | 30 |

The results show that method B appears to have a greater impact on the reduction of microorganisms, as the plate count is below 100. The plate count reduction in Method A is also substantial, but appears to be a bit more fluctuating. For that reason, Method B with an intermediate step for germination of surviving spores, followed by lytic enzyme incubation and cellulase and hemicellulase incubation is preferred. The solubilization yield was 40% for both of these methods.

### Example 5

### Pretreatment of Pig Manure at Pilot Scale

Pig manure concentrate was obtained from a manure trader. The composition of this material is presented in Table 6.

TABLE 6
Composition of pig manure concentrate, as expressed in g/kg.

| | Content (g/kg) |
|---|---|
| Dry matter | 301 |
| Organic dry matter | 223 |
| Ash | 78 |
| Protein | 48 |
| Ammonia—N | 5 |
| Lipids | 8 |
| Lignin | 88 |
| Carbohydrates | 67 |

Solubilized pig manure was prepared in a similar process set-up as described in Example 2. 205 kg of pig manure concentrate was transferred to the stainless steel tank reactor to which 400 L water was added. The slurry was mixed at 50-55 rpm and heated to 90-95° C., using 0.5 bar steam in the heating jacket. To adjust the pH of the slurry to pH 11, 13.5 kg of 25% NaOH was added, followed by incubation at 90-92° C. for 4 h, at a stirrer speed of 40 rpm. The pH had dropped to 8.5, and the slurry was cooled to 30-32° C., using cold water in the cooling jacket of the reactor. The pH was adjusted to pH 7 by addition of 27.3 kg 10% HCl for germination of potentially remaining bacterial spores, during incubation of 4 h at a stirring speed of 50 rpm. Subsequently, the temperature was increased to 60-62° C., the pH was adjusted to pH 8, using 2.7 kg 25% NaOH, and 8 kg of Delvolase® and 4 kg of Delvozyme® L was added. After 4 h incubation at 60-62° C. and a stirring speed of 25-30 rpm, the slurry was heated to 90° C. and incubated at that temperature for 3 h. The slurry was cooled to 50-52° C., the pH was adjusted to pH 4.5 by addition of 67.7 kg 10% HCl. 8 kg of solution containing 600 g of protein derived from Bakezyme® ARA10.000, and 8 kg of solution containing 720 g of protein derived from Filtrase® NL was added, and the slurry was incubated for 20 h at 50-52° C., while stirring at a speed of 25-30 rpm. Finally, the slurry was cooled to room temperature and the pH was adjusted to 3.5 by the addition of 13 kg 10% HCl. 25 kg of slurry was withdrawn, before the remainder was filtered in 2 cycles. The primary filtrates were combined and amounted to 496 kg, the 260 kg washing liquor was collected separately. The primary filtrate was used for subsequent anaerobic fermentation experiments. The composition of this primary filtrate is given in Table 7.

TABLE 7
Composition of pig manure primary filtrate, as expressed in g/kg.

| | Primary filtrate |
|---|---|
| Dry matter | 59 |
| Organic dry matter | 40 |
| Ash | 19 |
| Protein | 7 |
| Ammonia - N | 2 |
| COD | 41 |

During the process, small samples were taken for total aerobic plate count. Results of these analyses are shown in Table 8.

TABLE 8
Aerobic total plate counts at the end of the various steps in
the process, as expressed as colony forming units per g (CFU/ml).

| Process step | CFU/ml |
|---|---|
| pH temperature treatment | 130 |
| pH 7 30° C. | <10 |
| Lytic enzyme treatment | >1.0 · 10⁸ |
| Thermal treatment | 1.9 · 10⁴ |
| Carbohydrase treatment | 1.9 · 10² |
| pH reduction | <10 |
| Filtrate | 120 |

From these results it is clear, that bacterial growth in pig manure can be controlled by careful adjustment of thermal, pH and enzymatic process steps.

### Example 6

### Impact of Pretreatment on the Conversion of BSG and Pig Manure into Biogas

### Experimental Set-Up

Two substrates were used as examples for this patent. The pre-treatment has been described above and were tested in the following reactor configurations (Table 9):

TABLE 9
Reactor configuration and substrate tested

| Reactor | Substrate | Reactor type |
|---|---|---|
| 1 | Non-pretreated BSG | CSTR |
| 2 | Pre-treated BSG | CSTR |
| | suspension | |
| 3 | Filtered pre-treated BSG | CSTR |
| 4 | Filtered pre-treated BSG | SBR (CSTR with biomass retention) |
| 5 | Filtered pre-treated BSG | EGSB |
| 6 | Filtered pre-treated PM | SBR (CSTR with biomass retention) |
| 7 | Filtered pre-treated PM | EGSB |

Substrate number 1 (Brewer spent grains-BSG) was tested more extensively: both the soluble fraction (centrifugate or filtrate), the total solution (suspension) were tested, as well as the non pre-treated material in a continuous stirred tank reactors (5 L). To evaluate the maximum conversion rate of the centrifugate, this substrate form was also tested in SBR (CSTR with biomass retention) and in the EGSB (3.8 L). In the EGSB only a liquid fraction can be used. For substrate 2 (Pig manure-PM), only the centrifugate fraction of the pre-treated material was evaluated in both the SBR and in the EGSB.

The reactors had the same starting inoculum: 20% volume of anaerobic granular sludge from a full-scale reactor, purchased from potato waste water processing plant Germany (UASB), were operated at the same temperature 36±2° C., the pH was controlled at 7.2±0.3 with NaOH/H2SO4 (2M).

The superficial flow velocity in the EGSB was 8 m/h.

Stable operation was assumed to be reached after test results showing <10% deviation for three consecutive samples (sampling twice weekly). The tests consisted of the VFA in the effluent, soluble COD and methane yield.

The organic loading rate (OLR-g-COD/L.d) was then stepwise increased up to its maximum: good COD conversion (>60%) and no VFA accumulation.

### Results

The two substrates were tested: brewer spent grains (BSG) and pig manure (PM), which underwent the pre-treatment as described above. The composition of the substrate into each reactor is presented in Tables 3 and 6. The substrate was diluted in order to apply the desired organic loading rate (g-COD/L.d) and hydraulic retention time (HRT).

For the BSG material, the three fractions were tested: centrifugate or filtrate (soluble fraction of the pre-treated material), suspension (whole pre-treated material and the original material (non-pretreated). These were tested in a continuous system (CSTR, 5 L). The centrifugate was also tested in the SBR (CSTR including settling cycles, in order to allow a longer retention time of the suspended solids, including the biomass). In the EGSB (3.8 L) only the liquid fraction was tested. For the PM only the centrifugate fraction was tested both in an SBR and in the EGSB.

The results show that the pre-treatment increased the availability of the COD to the microorganisms, as the system can operate at higher conversion rates, up to 2 fold (Reactors 1, 2 and 3) with no accumulation of organic acids (VFAs) and good COD conversion (>60%).

The impact of the biomass (VSS) concentration inside the reactors is seen by comparing reactors 3 with 4 and 5. A higher biomass concentration (number of cells) allows a higher conversion of the organic load imposed and thereby a higher methane production rate, as well as a higher yield.

The effect of a better mixing and mass transfer between the solid-liquid-gas fractions can be seen by comparing reactors 4 and 5, which have a different configuration. Operating in an EGSB resulted in an increase of >50% of productivity compared to the SBR. The same effect is observed for the second substrate, PM, in reactors 6 and 7; the effect was higher than 100% under stable operation.

The combination of the pre-treatment, solid liquid separation and reactor configuration, showed advantageously the highest methane production rates (>3 fold) and 20% increase of the methane yield.

TABLE 10
Effect of pre-treatment (PT) and reactor configuration on methane yield and production rate

| | Reactor | | | | | | |
|---|---|---|---|---|---|---|---|
| | 1 | 2 | 3 | 4 | 5 | 6 | 7 |
| | Substrate | | | | | | |
| | 1-BSG | | | | | 2-PM | |
| | non-PT | PT susp | PT centrifugate | | | PT centrifugate | |
| Description | CSTR-1 | CSTR-2 | CSTR-3 | SBR | GSB | SBR | EGSB |
| OLR (g-COD/L · d) | 4.0 | 5.3 | 6.3 | 7.9 | 12.3 | 3.0 | 12.4 |
| OLR (g-oDM/L · d) | 3.0 | 4.0 | 4.5 | 5.6 | 8.8 | 3.3 | 13.6 |
| Factor to correct for the | 1.0 | 0.8 | 0.8 | 0.8 | 0.8 | 0.4 | 0.4 |
| Enzyme solution | | | | | | | |
| HRT (d) | 15 | 15 | 10 | 12.1 | 2.5 | 12 | 2.1 |
| Temperature (° C.) | 38 | 38 | 38 | 38 | 36 | 38 | 36 |
| pH | 7.2 | 7.5 | 7.4 | 7.7 | 7.1 | 7.2 | 7.1 |
| Volume (L) | 5 | 5 | 5 | 5 | 3.8 | 5 | 3.8 |
| VSS (g/L) | | | | | | | |
| inside the reactor | 24 | 8.5 | 15 | 27 | 20 | 17 | 12 |
| effluent | 24 | 8.5 | 15 | 16 | 1.9 | 9.6 | 7.1 |
| COD removal (%) | | | | | | | |
| total | n.d. | n.d. | 74 | 80 | 81 | 85 | 60 |
| soluble | n.d. | n.d. | 82 | 89 | 88 | 91 | 77 |
| VFA (acetic + propionic acid) | <0.3 | <0.3 | 0.6-1.0 | 0.6-1.2 | <0.3 | 0.6-1.0 | 0.6-1.0 |
| (g/L) | | | | | | | |
| methane yield (ml/g-COD in) | 153 | 150 | 196 | 193 | 188 | 83 | 53 |
| methane yield (ml/g-oDM in) | 205 | 258 | 295 | 292 | 282 | 140 | 88 |
| methane production rate | 617 | 802 | 1224 | 1521 | 2312 | 249 | 651 |
| (ml/L · d) | | | | | | | |

1. A process for digestion of organic material into biogas which comprises:
treating the organic material to reduce the number of viable microorganisms in the organic material;
treating the organic material with at least one enzyme;
separating a liquid fraction from a solid fraction of enzyme-treated organic material; and
digesting the liquid fraction to form biogas.

2. A process according to claim 1, wherein said organic material is treated at a temperature of from 65° C. to 120° C., optionally at a temperature of from 65° C. to 95° C.

3. A process according to claim 1, wherein said organic material is treated at low or high pH, optionally at pH<4, or optionally at pH>8.

4. A process according to of claim 1, wherein CFU count in the organic material after treatment to reduce number of viable microorganisms, is lower than 10⁶, optionally less than 10⁵ CFU/ml in the organic material present.

5. A process according to claim 1, wherein the at least one enzyme is selected from the group consisting of protease, lipase, lysing enzyme, phytase, hemicellulase and cellulase.

6. A process according to claim 1, whereby at least one thermostable enzyme is used.

7. A process according to claim 1, whereby a liquid fraction of stillage is digested in a biogas fermenter wherein a HRT in a EGSB digester of from 3 hours to 100 hours, a HRT in a IC reactor of from 3 hours to 100 hours, a HRT in an UASB digester of from 10 hours to 100 hours, a HRT in a CSTR digester of from 1 day to 20 days and/or a HRT in an anaerobic membrane bioreactor of from 3 days to 12 days is used.

8. A process according to claim 1, whereby a liquid fraction of stillage is digested in a biogas fermenter, which is an UASB, IC, anaerobic membrane bioreactor and/or EGSB reactor.

9. A process according to claim 1, whereby an organic material is treated with at least one enzyme, wherein said treating takes from 2 hours to 50 hours, optionally from 3 hours to 30 hours.
